(19) United States
(12) Patent Application Publication
(10) Pub. No.: US 2017/0143253 A1
(43) Pub. Date: May 25, 2017

(54) DEVICE, METHOD AND COMPUTER PROGRAM FOR DETECTING MOMENTARY SLEEP

(71) Applicants: Fraunhofer-Gesellschaft zur Foerderung der angewandten Forschung e.V., Munich (DE); Technische Universitaet Ilmenau, Ilmenau (DE)

(72) Inventors: Daniel KRENZER, Wutha-Farnroda (DE); Albrecht HESS, Schoenbrunn (DE); András KÁTAI, Ilmenau (DE); Matthias PAULIGK, Ilmenau (DE); Paul FRITZSCHE, Ilmenau (DE); Lautet Joachim TILGNER, Ilmenau (DE); Michael HAENSEL, Ilmenau (DE); Anja CHILIAN, Ilmenau (DE); Tamás HARCZOS, Wolfsberg OT Wuembach (DE); Johannes-Wolf KUENZEL, Ilmenau (DE); Peter HUSAR, Ilmenau (DE); Christian WIEDE, Chemnitz (DE)

(21) Appl. No.: 15/381,696

(22) Filed: Dec. 16, 2016

Related U.S. Application Data
(63) Continuation of application No. PCT/EP2015/056748, filed on Mar. 27, 2015.

(30) Foreign Application Priority Data
Jun. 20, 2014 (DE) 10 2014 211898.0

(51) Int. Cl. A61B 5/00; A61B 3/00; A61B 3/14; A61B 5/11; A61B 5/18

(52) U.S. Cl. CPC A61B 5/4809; A61B 5/1103; A61B 5/1128; A61B 5/18; A61B 5/746; A61B 3/145; A61B 5/0077; A61B 5/6893; A61B 5/7282; A61B 5/1114; A61B 5/1112; A61B 3/0025; A61B 2562/0219; A61B 2560/0431; A61B 2576/00; B60W 40/08

(57) ABSTRACT

A device for detecting momentary sleep is shown. The device includes a video recording device for video-based monitoring of a person and of an eye area of the person, the video recording device being configured to record a sequence of pictures of the person and of the eye area and to output same to a threshold determination device. The threshold determination device is configured to derive one or more thresholds individually adapted to the person from the sequence of pictures. Moreover, the device includes a threshold evaluator configured to decide, on the basis of the one or more individually adapted thresholds, whether or not the person has momentarily fallen asleep, the one or more individually adapted thresholds being utilized for establishing whether or not the thresholds have been passed within the actual time curve of the eye opening and, thus, whether or not the person has momentarily fallen asleep.

### CROSS-REFERENCE TO RELATED APPLICATIONS

This application is a continuation of copending International Application No. PCT/EP2015/056748, filed Mar. 27, 2015, which is incorporated herein by reference in its entirety, and additionally claims priority from German Application No. 102014211898.0, filed Jun. 20, 2014, which is incorporated herein by reference in its entirety.

The present invention relates to a device, a method and a computer program for detecting momentary sleep. Embodiments show a momentary-sleep warning device.

### BACKGROUND OF THE INVENTION

Driver assistance systems which also include momentary-sleep warning devices can already be found in some series-production vehicles. They are based on statistical evaluation of data available on a CAN bus, for example (e.g. steering angle, accelerating and breaking behavior, etc.). In other words, driver assistance systems which up to now have been available in series-production vehicles evaluate data allowing only indirect and imprecise conclusions to be drawn about the state the driver is in and, in particular, with regard to momentary sleep.

Moreover, it is possible to attach retrofitting systems directly to the person. Exemplary places of attachment are an ear or a hand. Thus, the system may react, e.g., to tilting of the head or to the conductance of the skin so as to recognize momentary sleep therefrom. Just like the above-mentioned driver assistance systems, said systems also have the disadvantage that the data evaluated allows only indirect and imprecise conclusions to be drawn about the state the driver is in and, in particular, with regard to momentary sleep. In addition, said systems are inconvenient to handle and unpleasant to wear by the user and are hardly accepted by potential users.

In addition it is possible to employ eyeglasses having integrated cameras which are directed at the eyes and are able to recognize momentary sleep via eyelid closure on a video basis. However, these systems, too, are inconvenient to handle and unpleasant to wear by the user and are hardly accepted by potential users.

Further known, camera-based momentary-sleep warning devices use, e.g., preset thresholds for eyelid closure determination. However, they are not very reliable since preset reference values are used for determining momentary sleep.

### SUMMARY

According to an embodiment, a device for detecting momentary sleep may have: a video recording device for video-based monitoring of a person and of an eye area of said person, the video recording device being configured to record a sequence of pictures of the person and of the eye area and to output same to a threshold determination device; the threshold determination device configured to derive one or more thresholds individually adapted to the person from the sequence of pictures; and a threshold evaluator configured to decide, on the basis of the one or more individually adapted thresholds, whether or not the person has momentarily fallen asleep; the one or more individually adapted thresholds being utilized for establishing whether or not the thresholds have been passed within the actual time curve of the eye opening and, thus, whether or not the person has momentarily fallen asleep; a first individually adapted threshold characterizing the transition from the opened to the closed eye, an eyelid closure being established to have occurred if the first individually adapted threshold is fallen below; a second individually adapted threshold referring to a duration of the eyelid closure, an individual eyelid closure time being determined so as to evaluate the eyelid closure as being an indication of momentary sleep if the individual eyelid closure time is exceeded.

According to another embodiment, a method of detecting momentary sleep may have the steps of: video-based monitoring of a person and of an eye area of said person by using a video recording device, said video recording device being configured to record a sequence of pictures of the person and of the eye area and to output same to a threshold determination device; deriving one or more thresholds individually adapted to the person from the sequence of pictures by using the threshold determination device; deciding, on the basis of the one or more individually adapted thresholds whether or not the person has momentarily fallen asleep; and utilizing the one or more individually adapted thresholds so as to establish whether or not the thresholds have been passed within the actual time curve of the eye opening and, thus, whether or not the person has momentarily fallen asleep; a first individually adapted threshold characterizing the transition from the opened to the closed eye, an eyelid closure being established to have occurred if the first individually adapted threshold is fallen below; a second individually adapted threshold referring to a duration of the eyelid closure, an individual eyelid closure time being determined so as to evaluate the eyelid closure as being an indication of momentary sleep if the individual eyelid closure time is exceeded.

According to another embodiment, a non-transitory digital storage medium may have a computer program stored thereon to perform the method of detecting momentary sleep, which method may have the steps of: video-based monitoring of a person and of an eye area of said person by using a video recording device, said video recording device being configured to record a sequence of pictures of the person and of the eye area and to output same to a threshold determination device; deriving one or more thresholds individually adapted to the person from the sequence of pictures by using the threshold determination device; deciding, on the basis of the one or more individually adapted thresholds whether or not the person has momentarily fallen asleep; and utilizing the one or more individually adapted thresholds so as to establish whether or not the thresholds have been passed within the actual time curve of the eye opening and, thus, whether or not the person has momentarily fallen asleep; a first individually adapted threshold characterizing the transition from the opened to the closed eye, an eyelid closure being established to have occurred if the first individually adapted threshold is fallen below; a second individually adapted threshold referring to a duration of the eyelid closure, an individual eyelid closure time being determined so as to evaluate the eyelid closure as being an indication of momentary sleep if the individual eyelid closure time is exceeded, when said computer program is run by a computer.

Embodiments show a device for detecting momentary sleep. The device comprises a video recording device for video-based monitoring of a person and of an eye area of said person, the video recording device being configured to record a sequence of pictures of the person and of the eye area and to output same to a threshold determination device. The threshold determination device is configured to derive one or more thresholds individually adapted to the person from the sequence of pictures. Moreover, the device includes a threshold evaluator configured to decide, on the basis of the one or more individually adapted thresholds, whether or not the person has momentarily fallen asleep; the one or more individually adapted thresholds being utilized for establishing whether or not the thresholds have been passed within the actual time curve of the eye opening and, thus, whether or not the person has momentarily fallen asleep. Passage may mean reaching as well as exceeding and/or falling below of a threshold. Thus, it is useful, for example, to determine reaching or falling below of the first threshold (degree of eyelid closure) and reaching or exceeding of the second threshold (period during which the first threshold is fallen below, e.g. period during which the eyelids are recognized as being closed). The sequence of pictures may further be processed in real time.

The invention is based on the finding that in particular with regard to the person, contactless detection of momentary sleep is possible by directly measuring eyelid closure, which is a good indicator of the fact that momentary sleep has occurred. Eyelid closure (or opening of the eyes) is determined, in this video-based process, by means of a predefined model, said model being individually adapted to the person. To this end, one might use a basic model of a head that is individually adapted to the person to be monitored. Measuring eyelid closure as a direct feature of momentary sleep is superior to measuring indirect features due to the very fact that no large-scale and, consequently, error-prone algorithms need to be applied to any indirect features such as tilting of the head or conductance of the skin. Moreover, in terms of convenience and acceptance, contactless measurement is clearly superior to measurement involving contact. A device mounted in a car, for example, requires no further adaptation (e.g. calibration on the part of the user), so that a person monitored will not notice at all that he/she is being recorded by cameras. This is not the case with specific eyeglasses, for example. They are to be put on before starting the journey and will permanently give the person a different feeling during the entire wearing period. This psychological effect substantially contributes to the user's wellbeing.

The criteria and/or thresholds used for detecting momentary sleep from any eyelid closure observed (and/or for individually adapting the model to the person) may be determined on the basis of monitoring the person and may thus be individually adapted to the person. The advantages here are that the person's anatomy and physiology are taken into account and that consequently, momentary sleep can be detected in a way that is considerably better than that using general standard values applied to the criteria and/or thresholds. Criteria individually adapted to the person's anatomy and physiology have improved validity with regard to the individual person as compared to general standard values which are determined, e.g., statistically across large groups of persons. This improved validity significantly increases the reliability of individual warnings in the event of momentary sleep.

Embodiments show a first individually adapted threshold, which is the eyelid closure of one eye, said eyelid closure lying between a first individually determined reference value describing an opened eye and a second individually determined reference value describing a closed eye, and the threshold characterizing the transition from the opened to the closed eye, a second individually determined threshold relating to a duration of the eyelid closure.

Embodiments further show that the threshold evaluator configured to detect the first threshold being passed and to determine the duration of said passing; momentary sleep being ascertained when said duration passes the second threshold. According to that, momentary sleep is recognized when the first threshold (e.g. while taking into account a hysteresis) is fallen below or reached, which means that closed eyelids have been recognized, and when the time period during which the eyelids are closed reaches or exceeds the second threshold.

Further individually adapted thresholds relating to further parameters and/or quality measures may be determined and taken into account, as criteria of the fact that the person monitored has momentarily fallen asleep, in evaluating the individually adapted thresholds. Utilization of such thresholds may facilitate, e.g., recognition of momentary sleep in that parameters such as a position, orientation or a quantity of distinctive patterns or points are determined. The distinctive patterns or points may be, e.g., the person's head, face, eyes, iris, pupil or pupil center. The further examples of individually adapted thresholds may be used individually or in any combination, for example also along with the above-mentioned individually adapted thresholds (degree of eyelid closure and duration of the eyelid closure). Also, they may replace the above-mentioned individually adapted thresholds (degree of eyelid closure and duration of eyelid closure).

According to further embodiments, the threshold determination device is configured to continually adapt the individually determined reference values and a first or several individually determined thresholds on the fly by evaluating any blinks that may occur. This is advantageous since the duration of eyelid closure, e.g. during winking, may change during the monitoring period, for example due to tiredness or the onset of dusk. Therefore, the threshold determination device may determine winking and/or deliberate or inadvertent spontaneous blinking by means of a pattern occurring during the time curve of the eye opening, said pattern comprising, from the direction of the first reference value, falling below the threshold as well as a return in the direction of the first reference value within a predefined time period. In addition, the threshold determination device may be configured to adapt the one or more individually adapted thresholds to the changed ambient conditions on the basis of a change in the ambient brightness. This is advantageous since consequently, rapidly changing eyelid closure characteristics, for example due to driving into a tunnel or to suddenly being dazzled by sunlight, can be taken into account in determining the individually adapted thresholds.

Embodiments further show the threshold determination device configured to adapt the one or more individually adapted thresholds to the driver's anatomy and physiology. Thus, e.g., thresholds individually determined for each person may be taken into account in determining momentary sleep. As long as the device has not determined, after having been switched on, any individual reference values or thresholds for the person, use may also be made of predefined, more general values so that the device nevertheless is able to operate during this time already.

Furthermore, the first individually adapted threshold may comprise a hysteresis according to which eyelid closure is defined at a relatively small eye opening angle during transition from an opened to a closed eye and is defined at a relatively large eye opening angle during transition from a closed to an opened eye.

Further embodiments show the threshold determination device determining a head pose and wherein the individually adapted thresholds are adapted by means of the head pose determined. This can be advantageously employed for rendering the continually determined eye opening curve quantitatively comparable for different head poses since the surface area of the eye that is visible to the device will change when the head is turned and will therefore seem smaller, for example, when the head is tilted forward. Moreover, the threshold determination device may be configured to determine the one or more individually adapted thresholds separately for each eye, whereby, e.g., that eye which can be more easily detected by the device will be used for momentary-sleep recognition. Also, the threshold determination device may be configured to determine, on the basis of the head pose, the eyelid closure on the basis of both said person's eyes, e.g. if both eyes are easily visible to the device, or to determine the eyelid closure on the basis of one eye of said person if, e.g., only one eye is visible to the device.

Further embodiments describe the device which includes a 3D modeler which calculates the position of the person's head pose on the basis of a three-dimensional model of a head. This is advantageous for determining a defined area in which the eyes are located. This reduces the number of detection errors and accordingly enables improved detection quality. In addition, the head pose may be taken into account in support of detecting eyelid closure. Depending on the location of the head in relation to the camera, the reference values for an open and/or closed eye may be corrected.

In accordance with a further embodiment, the 3D modeler may calculate an estimation of the three-dimensional model of the head, e.g., on the basis of a cylinder and may track it by means of feature tracking. Said estimation reduces the computing expenditure for detecting the head pose while providing a very good estimation for further determination of the eye areas. This enables analyzing (tracking) the head pose in “real time”.

Embodiments further show that the second individually adapted threshold indicates a duration longer than that of a wink. In addition, the second individually adapted threshold may be adapted to the current speed at which the person is moving, e.g. with a vehicle. For determining the current speed at which the person is moving, the device may have, e.g., a GPS receiver and/or an acceleration sensor configured to capture a speed of travel of the device, which may be used for drawing conclusions as to the person's and/or the vehicle's speed. The threshold determination device 20 is further configured to adapt the one or more individually adapted thresholds 80 and 85 to the speed of travel. This enables switching off of the momentary-sleep recognition and/or of the momentary-sleep warning when the person is sitting in a vehicle, for example, but said vehicle does not move. In a further embodiment, the device may also receive the speed of travel as an input signal.

According to further embodiments, the device comprises a source of illumination configured to emit radiation above a wavelength range visible to the person, the video recording device being configured to detect the radiation. This is advantageous in order to uniformly illuminate the person (even with changing external light conditions) without the person noticing the illumination and being disturbed by it. Moreover, the device may comprise a further source of radiation arranged at a distance from the source of radiation, the video recording device being configured to create a combination of pictures taken wherein the person is illuminated in that the eye area is successively illuminated by the source of radiation and the further source of radiation, so as to avoid or reduce reflections occurring in the eye area of the person. What may be advantageous about this arrangement is the improvement in the picture quality and, consequently, the accuracy of momentary-sleep determination since without avoiding or reducing reflections, reflections occurring on eyeglasses or on a pupil, for example, may obscure one or both said person's eyes and may thus render momentary-sleep determination more difficult.

According to further embodiments, the device includes an eye opening detector configured to determine eye opening parameters on the basis of the sequence of pictures. Embodiments show the eye opening detector configured to determine eye opening (e.g., eye opening normalized to the distance from the video recording device or a camera) on the basis of parameters from eye detection. The parameters may be the center of the eye or points in the edge area of the eye, for example. Since the eye opening degree of an eye is determined already within a limited region of the eyes, the determination is more accurate and less prone to errors than it would be if it were performed within a larger search area, or a larger ROI (region of interest).

Embodiments further describe determining the eye opening angle by adapting an eye template, or a model, to a gradient picture or other suitable features such as corner features or arch features, for example, which may be based on a combination of gradients in a specific constellation and/or distance in relation to one another. The eye template may be used for describing parts of an eye opening process. A further embodiment shows determining of the eye opening angle by means of an eye template, the eye template being spanned, by means of estimation, with a horizontal projection function on the basis of the center of the eye and a width of a face. In addition to this, an embodiment shows determining of the eye opening process by means of a curve approximation. The curve approximation may interpolate between at least four parameters describing the eye contour and may thus adapt an eye template to the detected eye. The described methods of determining the eye opening process use the parameters from the eye detection as reference points (=landmarks). The latter influence the sizes and shapes of the templates and therefore form the basis for determining the eye opening and/or the degree of eyelid closure.

In accordance with an embodiment, the device is portable. This enables mobile utilization of the device without same being fixedly installed in a car, for example.

Moreover, embodiments show a method of detecting momentary sleep which may be implemented as a computer program.

### BRIEF DESCRIPTION OF THE DRAWINGS

Embodiments of the present invention will be detailed subsequently referring to the appended drawings, in which:

FIG. 1 shows a schematic block diagram of a device for detecting momentary sleep;

FIG. 2 shows a schematic representation of an eye opening curve with individually determined reference values as well as individually adapted thresholds for detecting momentary sleep;

FIG. 3 shows a schematic block diagram of a device for detecting momentary sleep in a representation deviating from FIG. 1 and comprising more detailed functional blocks;

FIG. 4 shows a schematic representation of a video recording device directed at a person;

FIG. 5 shows a schematic representation of the interior of a vehicle with exemplary arrangements of the device for detecting momentary sleep; and

FIG. 6 shows a schematic flowchart of a method of detecting momentary sleep.

### DETAILED DESCRIPTION OF THE INVENTION

In the following description of the figures, elements which are identical or have identical actions will be provided with identical reference numerals, so that their descriptions are interchangeable in the different embodiments.

Overtiredness in persons (e.g. drivers of vehicles) may result in decreasing attentiveness and in the person falling momentarily asleep. Momentary sleep here will be used as a synonym for an occurring brief period of sleep and also includes microsleep, for example. This causes about 25% of fatal accidents on German motorways. Currently, no system is known on the market which provides reliable warning in the event of momentary sleep. Therefore, a device for detecting momentary sleep will be presented below which accurately and reliably detects eyelid closure in a person so as to conclude the person's momentary sleep therefrom, which detects momentary sleep in a person, outputs a warning when momentary sleep occurs, and enables easy usability of the system for providing momentary-sleep warning.

The overall system may derive individually adapted thresholds for detecting momentary sleep on the part of the person to be monitored from a camera picture in which the face of the person is pictured, and warns the person when momentary sleep occurs. For reasons of improved clarity, mention is made of a driver in some embodiments. However, the description and the type of application of the invention by analogy also relate to further fields of application, e.g. monitoring of aviation areas by air traffic controllers, in which case the system may be used for emitting a warning in the event of momentary sleep on the part of an air traffic controller.

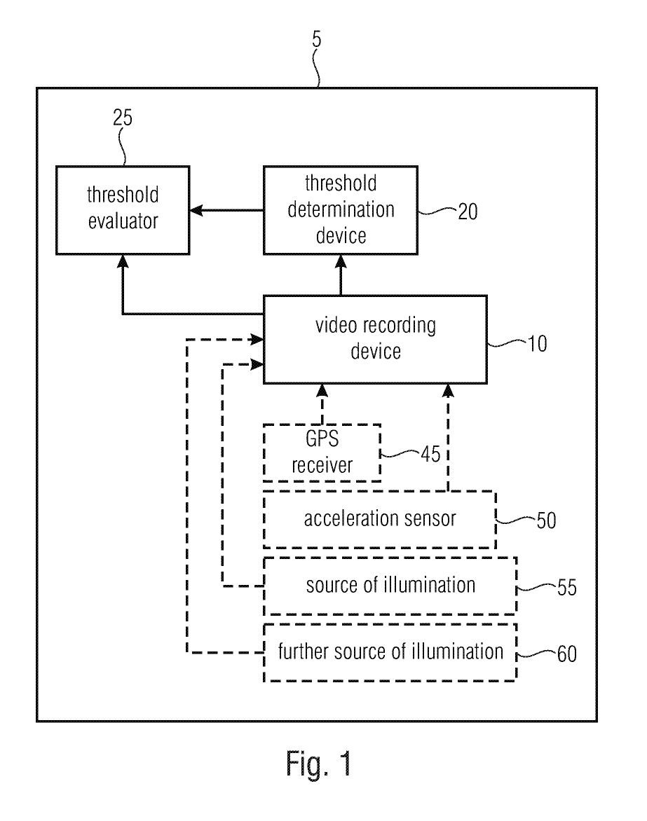

FIG. 1 shows a schematic block diagram of a device 5 for detecting momentary sleep. The device 5 for detecting momentary sleep comprises a video recording device 10, a threshold determination device 20 as well as a threshold evaluator 25. The video recording device 10 for video-based monitoring of a person 15 and of the eye area of the person 15 is configured to record a sequence of pictures of the person 15 and of the eye area and to output same to the threshold determination device 20. The threshold determination device 20 is configured to derive one or more thresholds 80 and 85, individually adapted to the person, from the sequence of pictures. The threshold evaluator 25 is further configured to decide, on the basis of the individually adapted thresholds, whether said person has momentarily fallen asleep, the individually adapted thresholds being used to establish, within the actual time curve of the eye opening, whether any thresholds have been passed (e.g. exceeded and/or fallen below) and, thus, whether the person has momentarily fallen asleep. Moreover, the threshold evaluator 25 may be configured for establishing momentary sleep in accordance with the above description.

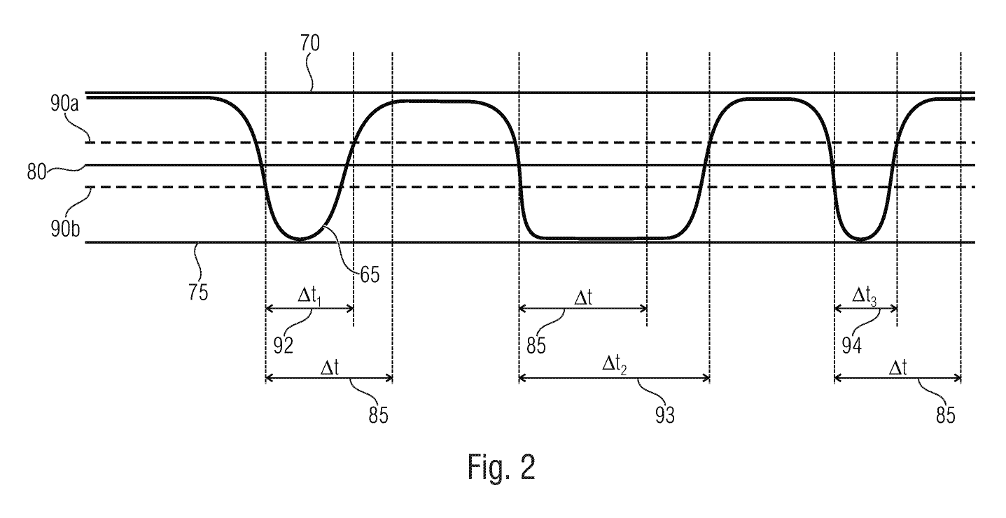

The threshold determination device 20 thus is configured to derive, from the sequence of pictures, time-variable thresholds for detecting momentary sleep which are individually adapted to the user, or the person, 15 and to ambient influences. Ambient influences are understood to mean, in particular, changing light conditions such as sun, shade, overcast sky, night or dazzling headlights of an oncoming vehicle, for example. In order to take into account the ambient influences in determining the individually adapted thresholds, the threshold determination device is configured to adapt the individually adapted thresholds to the changed ambient conditions on the basis of a change in the ambient brightness. The thresholds are intended to describe a “time curve of eye opening” characteristic for momentary sleep in that one threshold defines which eye opening is considered as being a closed eye, and the other threshold defines for how long the eye may be recognized as being closed for momentary sleep to be established. The thresholds will be explained in more detail below with reference to FIG. 2.

The individually adapted thresholds 80 and 85 may take into account features, e.g. anatomical or physiological features, specific to the person 15. From the individually adapted thresholds 80 and 85, the threshold evaluator 25 may determine the occurrence of eyelid closure as an indicator of the onset of momentary sleep. This will be explained below in more detail.

Optionally, the device 5 may comprise a GPS receiver 45 and/or an acceleration sensor 50. The sensors may sense a speed of travel of the device 5, which may be used as a basis for drawing conclusions as to the speed of the person and/or of the vehicle. The threshold determination device 20 is further configured to adapt the individually adapted thresholds 80 and 85 to the speed of travel. For example, in the case of utilization in a vehicle, the speed and/or the driving condition (moving/being stationary) of the vehicle may be recognized and the momentary-sleep warning may be switched off since momentary-sleep warning for a driver may advantageously be used in a moving vehicle or, generally, in a moving driver.

Further embodiments show the device 5 with a source of illumination 55 which emits radiation above a wavelength range visible to the person 15, the video recording device 10 being configured to detect the radiation. Thus, the person 15 may be illuminated independently of the external light conditions, e.g. also in complete darkness, without the person 15 being dazzled or disturbed.

Optionally, the device 5 may also comprise a further source of illumination 60 arranged at a distance from the source of illumination 55. Thus, the person 15 may be illuminated by two sources of illumination 55 and 60 at different angles. The video recording device 10 is configured to determine a combination of pictures taken wherein the person 15 is illuminated by subsequent illumination of the eye area by means of the source of illumination 55 and the further source of illumination 60, so as to avoid or reduce any reflections in the region of the person's eye area.

FIG. 2 shows a schematic representation of a curve of the eye opening 65 with individually determined reference values 70 and 75 as well as the individually adapted thresholds 80 and 85 for detecting momentary sleep. Eye opening may be an absolute eye opening or an eye opening normalized, e.g., to a distance of the eye from the video recording device 10. The first individually adapted threshold 80 is used for determining eyelid closure of one eye, the eyelid closure being defined to lie between the first individually determined reference value 70, which describes an opened eye, and the second individually determined reference value 75, which describes a closed eye, and the threshold 80 characterizing the transition from the opened to the closed eye (eyelid closure). In other words, eyelid closure will be established when a certain degree of the eye opening, which is individually adapted to the user, is due to the ambient influences, and evaluates the eye as being closed, is fallen below.

The second individually adapted threshold 85 relates to a duration of the eyelid closure. An individual eyelid closure time is determined so as to evaluate the eyelid closure as being momentary sleep if said eyelid closure time is exceeded. Momentary sleep will be established when both individually adapted thresholds 80 and 85 for the eyes used for evaluation are fallen below (eye opening) or exceeded (time threshold). The threshold determination device 20 accordingly is configured to determine blinking and/or winking by means of a pattern within the time curve of the eye opening 65; the pattern coming from the direction of the first individually determined reference value 70 comprises falling below the first individually adapted threshold 80 and a return in the direction of the first reference value 70 within a predefined time Δt. Depending on the detectability of the eyes by the video recording device 10, one eye or both eyes is/are used for determining momentary sleep. This will be explained in more detail below.

Further individually adapted thresholds relating to further parameters and/or quality measures may be determined and taken into account, as criteria of the fact that the person monitored has momentarily fallen asleep, in evaluating the individually adapted thresholds. Utilization of such thresholds may facilitate, e.g., recognition of momentary sleep in that parameters such as a position, orientation or a quantity of distinctive patterns or points are determined. The distinctive patterns or points may be, e.g., the person's head, face, eyes, iris, pupil or pupil center. The further examples of individually adapted thresholds may be used individually or in any combination, for example also along with the above-mentioned individually adapted thresholds (degree of eyelid closure and duration of the eyelid closure). Also, they may replace the above-mentioned individually adapted thresholds (degree of eyelid closure and duration of eyelid closure).

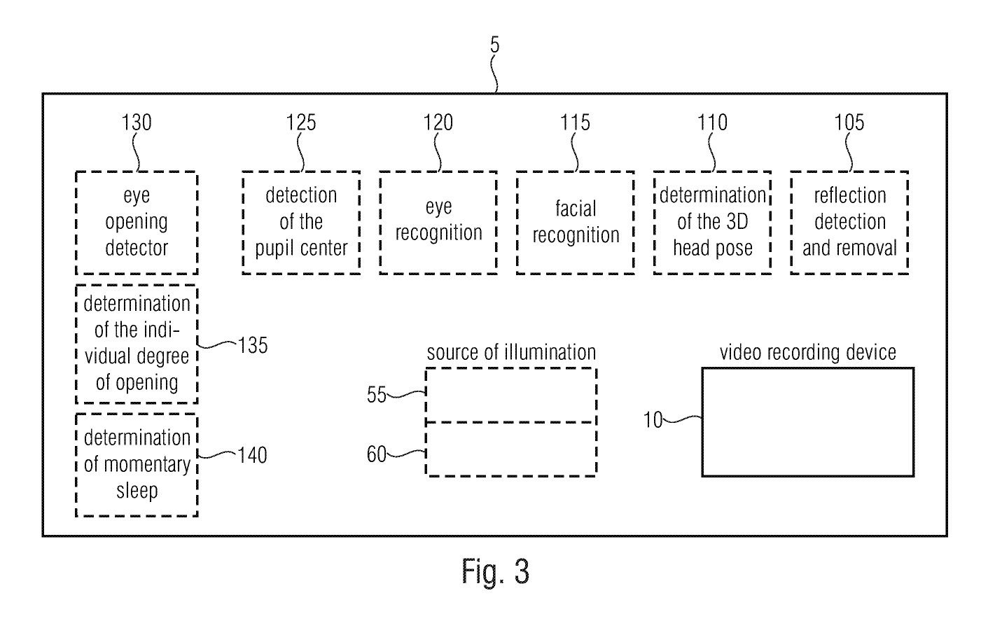

For determining the first individually adapted threshold 80, eye opening is measured and eyelid closure is detected. This is performed separately for each eye. A method of measuring eye opening will be described with reference to FIG. 3. The result is a time curve of the eye opening 65 (cf. FIG. 2), expressed in absolute or normalized values. By means of the normalization, for example as pixels normalized to the distance of the person's 15 monitored eye from the camera 10, the influence of different distances between the driver and the device may be eliminated.

The first individually determined reference value 70, also referred to as the upper reference value and describing an opened eye, and the second individually determined reference value 75, also referred to as the lower reference value and describing a closed eye, may be determined from the time curve of the eye opening 65 inclusive of any winking and/or blinking. Moreover, the first individually adapted threshold 80 may be determined within the band limited by the individually determined reference values 70 and 75. The eyelid is considered as being closed (eyelid closure) when the current eye opening is smaller than the first individually adapted threshold. According to this, the threshold determination device is configured to adapt the individually adapted thresholds to the driver's anatomy and physiology. In addition, the individually determined reference values 70 and 75 and the individually determined thresholds 80 and 85 are continually adapted on the fly on the part of the threshold determination device 20 by means of evaluating any blinking which occurs.

Moreover, the first individually adapted threshold 80 may comprise a hysteresis 90a, 90b configured to define eyelid closure at a relatively small eye opening angle 90b during transition from an opened to a closed eye, and to define eyelid closure at a relatively large eye opening angle 90a during transition from the closed to the opened eye.

The individually determined reference values 70 and 75 for an open and/or closed eye, individually concerning the person 15 and the ambient conditions, are constantly determined by evaluating the monitored person's blinking, which may be spontaneous or inadvertent, for example. This is advantageous since in this manner, individual anatomical and physiological features of the person 15 are taken into account. Furthermore, the method is resistant to any disturbances caused by ambient influences on the monitored person 15, e.g. dazzling light, for example, since the thresholds and/or reference values are dynamically adapted and therefore adapt to any situations that may have changed. Said evaluation may be effected on the basis of recognizing spontaneous blinking or winking, “open” and “closed” states of which are considered as being an individually open and/or closed eye.

Subsequently, a method of recognizing winking or blinking will be explained by way of example. However, further methods may also be employed. Once a blinking process has terminated, same will be recognized, by means of the signal shape in the time curve of the eye opening 65, as a pattern that is variable within certain limits. The pattern includes a reduction in the eye opening, followed by enlargement of the eye opening approximately to the initial value of 70, both of which occur in close succession. The maxima and minima of said waveforms, which may be filtered in order to improve their capacity to be evaluated, are used as the upper and lower reference values 70 and 75.

In accordance with a further embodiment, the threshold determination device may determine the individual reference value for an individually “opened” and an individually “closed” eye, respectively. These two individual reference values describe the eye as being individually “open” or “closed”. To perform said determination, an individual tendency of these two reference values is recorded, i.e. one continually captures what exactly said individually “opened” (corresponds to the first individually adapted reference value 70) and said individually “closed” (corresponds to the second individually determined reference value 75) of said person 15 amounts to, and the first individual threshold 80 is defined to lie between both said individual reference values (eyelid closure). In other words, a closed eyelid is defined in terms of percentage by said being individually “open” or “closed”.

Individual momentary sleep may be determined from said individual thresholds. The adaptive threshold 80 which is determined in advance and as of which the degree of eye opening is evaluated as being critical is supplemented by a hysteresis 90a, 90b. The threshold is adaptive since it relates to the individual reference value for the individually “opened” and “closed” eye. This principle will be explained in more detail with reference to FIG. 3.

For fine-adjusting the first individually adapted threshold 80, the eyelid-closure speed may also be taken into account. Said eyelid-closure speed may be determined, for example, by means of the duration which starts with leaving the first individually determined reference value 70 and ends with reaching the second individually determined reference value 75. If the individually adapted threshold 80 is positioned halfway between the upper and lower reference values 70 and 75, for example, the eyelid-closure speed may be exploited in that the individually adapted threshold 80 is corrected in the upward direction (toward reference value 70) or in the downward direction (toward reference value 75).

In accordance with a further embodiment, reference values 70 and 75 are also fine adjusted. This is effected while taking into account the head pose, i.e. the position and/or spatial location and orientation of the head, which may be determined by means of so-called head-pose trackings. According to this, the threshold determination device 20 is configured to adapt the individually adapted threshold 80 by means of the determined head pose and to determine the eyelid closure by means of one eye or both eyes of the person 15 as a function of the head pose. In particular the first individually determined reference value may be determined in an imprecise manner due to a changed perspective in which a picture of the eye was taken, for example if the center of the eye, in which the eye opening is largest due to the curvature of the eyelid, is hidden from view to the video recording device 10. By means of said head-pose tracking one may therefore determine whether the look of the person 15 is frontally directed at the video recording device 10 or whether a correction of the reference values 70 and 75 may alternatively be performed. According to this, the head pose may be used in support of momentary-sleep recognition and may thus optionally be employed for improving eye evaluation.

On the basis of the evaluation of the recorded video data, a 3D head model may be adapted to the shape, position and orientation of the monitored head, and on the basis of the knowledge of the shape, position and orientation, the thresholds may be individually adapted. According to this, the device 5 may include a 3D modeler which calculates a three-dimensional model of the person's head on the basis of an evaluation of the individually adapted thresholds. To simplify things, the 3D modeler may also perform calculation by using a simplified representation of the shape, position and orientation of the three-dimensional model of the head, e.g. on the basis of a cylinder. Head-pose tracking will be described in more detail with reference to FIG. 3.

The second individually adapted threshold 85, i.e. a time period Δt of the eyelid closure, is determined and used for both eyes together, provided that both eyes are within focus and can be evaluated. The threshold 85 is determined dynamically on the basis of an individually determined time maximum of the eyelid closure duration; the eyelid closure duration may be determined, for example, by a multiple of the time duration of spontaneously occurring blinking or winking (characterized by an eyelid closure and an immediately following eyelid opening) or on the basis of a literature value. The literature value may serve as a reference point for not defining the value to be unrealistically high, or may serve as an initialization value for the event that the individual eyelid closure time has not yet been determined. Optionally, the second individually adapted threshold 85 may also depend on the person's speed of travel, for example in a car. For example, a relatively long eyelid closure time is more dangerous at a high speed than at a low speed since a larger distance is covered with closed eyes during the same time period. The data of the GPS receiver 45 and/or of the acceleration sensor 50 may be used for this purpose. Moreover, the time minimum of the second individually adapted threshold 85 may be adapted to the time period of the occurring blinking and/or winking.

The second individually adapted threshold 85 is indicated, within the band, to lie between the above-described maximum and minimum and may be adjusted as a function of the sensitivity that is set with regard to momentary-sleep warning. Given a high level of sensitivity (with specificity being low at the same time), the threshold will be close to the time minimum, and given a low sensitivity (with specificity being relatively high at the same time), the threshold will be close to the time maximum. The adjustment may be selected in the range from “high sensitivity” to “low sensitivity” as need be. A high level of sensitivity is useful, for example, in case the person is traveling fast.

By way of example, FIG. 2 shows the eye opening curve 65 within the limits of the individually determined reference values 70 and 75. The adaptive threshold 80 is defined to be halfway between the individual reference values and determines the eyelid closure of the person 15; in simplified terms, one speaks of an eyelid closure when the eye opening curve falls below the threshold 80. Around the threshold 80, the hysteresis range is defined between the limits 90a and 90b, which hysteresis range determines the starting and finishing times of the eyelid closure and, thus, the eyelid closure duration. If the limit 90b is fallen below, the eye will be considered as being closed, and if the limit 90a is exceeded, the eye will be evaluated as being opened. If the eyelid closure duration exceeds the defined threshold 85 (Δt), momentary sleep has occurred, otherwise what is at hand is winking and/or spontaneously occurring eyelid closure.

In other words, the individually adapted thresholds 80 and 85 define a pattern compared to the actual eye opening curve for detecting momentary sleep so as to detect momentary sleep in the event of there being a match between the pattern and the actual eye opening curve (which corresponds to the thresholds being exceeded and/or fallen below). With regard to FIG. 2, a timer will be started for determining the second adapted threshold 85 if the second hysteresis limit 90b is fallen below. Said timer will run for such time until the first hysteresis limit 90a is exceeded again. If the duration between (an initial) falling below and a (subsequent) exceeding of the hysteresis thresholds is shorter than the second threshold 85 (Δt), what is at hand is a spontaneously occurring eyelid closure or winking, as is shown by exemplary time periods Δt₁ 92 and Δt₃ 94. If the duration of the falling below and/or exceeding is larger than or equal to the second individually adapted threshold 85, what is at hand is momentary sleep. This is shown by means of the exemplary eyelid closure having the duration Δt₂ 93.

In other words, momentary sleep will occur if (in simplified terms) both eyelids are closed at a defined degree x for a defined time period Δt. Depending on the head's position, e.g. if only one of both eyes is captured by the video recording device 10, evaluation is also possible on the basis of this one eye. Moreover, detection of momentary sleep should be available immediately once the evaluation has started. To this end, a relatively large degree of uncertainty and/or a large degree of sensitivity may be accepted since there will be few or no individual data about the monitored person available at the beginning of a measurement.

For deriving the described detection thresholds 80 and 85 and the reference values 70 and 75, individual anatomical and physiological features (for example individual anatomical eye opening) and individual parameters such as eyelid closure, head inclination etc. will be used.

Moreover, the 3D head pose may be used for defining which of the two eyes is used, or whether possibly both eyes are used, for evaluation. Depending on the position and twisting of the head in relation to the camera, a decision will be made about which of the two eyes, and whether possibly both eyes, will be used for detecting momentary sleep. If head pose tracking determines sufficiently frontal orientation of the head with regard to the camera, both eyes will be used for evaluation.

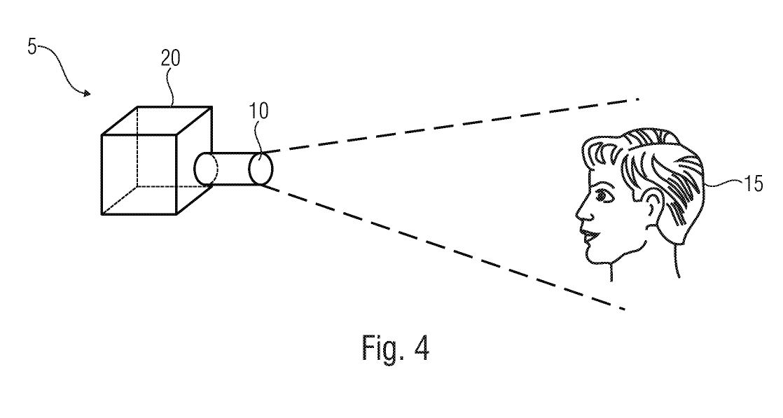

FIG. 3 shows a schematic block diagram of a device 5 for detecting momentary sleep in a representation which deviates from FIG. 1 and comprises more detailed functional blocks. The device 5 for detecting momentary sleep includes a video recording device 10 for video-based monitoring of a person 15 and of an eye area of said person 15. Moreover, the device 5 for detecting momentary sleep optionally includes a source of illumination 55 and, optionally, further functional blocks 105-140. Depending on the implementation, the functional blocks may be associated with the threshold determination device 20, the threshold evaluator 25 or a separate computing device (not shown). According to embodiments, the device 5 is contactless with regard to the person 15 (see FIG. 4). Contactless means that when the device 5 is used as intended, no part thereof will be in contact with the person 15.

The video recording device 10 is configured to record a sequence of pictures of the person 15 and of the eye area and to output same to the functional blocks so as to decide whether or not the person 15 has momentarily fallen asleep.

The video recording device 10 further is configured to record the person 15 or at least a face of the person 15 and to output a sequence of the pictures taken to the functional blocks 105-140. On the basis of said sequence of pictures, the functional blocks will perform picture recognition and processing steps.

Embodiments show the distance of reflections 105 in the sequence of pictures. Reflections may occur, e.g., on eyeglasses or the pupils and/or the eyes (in particular on the cornea). Reflections may be removed algorithmically by detecting and segmenting the reflection and filling it up with interpolated or, if possible, reconstructed picture content. Moreover, reflections may be avoided in that the person is alternatingly illuminated with light, e.g. from the infrared spectrum, from two different positions. Two successive frames may thus be calculated in relation to each other such that the reflections are clearly reduced since the arising reflections occur at different picture positions due to the variation in illumination. A further embodiment describes a modification of said latter algorithm. Again, two light sources, e.g. infrared LEDs (IR-LEDs), are alternatingly used for illumination, and reflections are continually detected, which reflections need not be corrected and/or interpolated. If the reflections are located within a region of interest (ROI), for example above the eye, the other existing light source or IR-LED may be used. In this manner, one may constantly switch between the light sources, and the illumination most advantageous in each case may be selected.

Embodiments show facial recognition 115. Facial recognition 115 may locate faces or components of a face, such as eyes, nose, mouth, ears and/or general points (e.g. landmarks) in a face or on a facial contour, for example by means of a cascade detector, so as to determine a two-dimensional (2D) position and size of the face as well as positions of the eyes (or other facial components). A cascade detector, e.g. according to Viola/Jones [2], may include different signal processing algorithms and/or identical signal processing algorithms having different levels of detail and/or levels of training so as to evaluate the general features. Utilization of hair-like features, shown in Viola Jones [2], for example, is optional in the embodiment described. In this manner, preclassification may be performed by means of simple algorithms, so that more specific algorithms need to be employed to a reduced data set only and so that, consequently, the computing time is reduced. Further, other signal processing methods such as neuronal networks, for example, may also be used instead of the cascade detector.

Further embodiments show eye recognition 120 being performed on the driver, or the person 15. The above-described face recognition may be refined in this step in that an analyzed 2D position, e.g. the center of the eyes, is determined with the eyes opened or closed. Moreover, an estimation regarding opening of the detected eyes may be performed. Again, eye recognition may be performed by using a cascade detector.

A further embodiment describes detection of the pupil, or the pupil center, 125. In particular with the eyes being opened, the pupil center may be determined and will then refine the position starting from the eye recognition 120. The pupil, or the pupil center, may be determined by means of a gradient-based method, for example [1]. The dark pupil and the iris, which is also dark, stand out clearly from the white eyeball and thus form a strong gradient which may be detected, e.g., by means of the method described in [1].

Additionally or alternatively, embodiments show the device 5 comprising a 3D head modeler for determining the 3D head pose 110, which calculates a three-dimensional model of the person's head, or a 3D head pose. By additionally taking into account the 3D head pose, a more robust and more accurate detection of the head, or the head position, may be achieved. In addition, landmarks in the ROI of the eye(s) for determining the degree of eye opening may be determined by means of a 3D model. Furthermore, in the eyelid evaluation, the tilting of the head may be taken into account just as much as the turning of the head so as to establish which eye can be evaluated more reliably. The tilting of the head may further be used for adapting the individual reference values 70 and 75 and the adaptive threshold 80. Thus, the device 5 is configured to determine a position of the eye of the person 15.

For determining the 3D head pose, initialization may take place at first. The latter includes a coarse 2D facial detection by means of a cascade detector (see facial detection 115). Further facial components may also be located via a cascade detector, as was already described for facial and eye recognition. Said components may be the position of the eyes, the tip of the nose, or the corners of the mouth. On the basis of the coarsely classified facial components, a 2D lattice network model may be placed or put over the face and be adapted to the person 15 by means of a method based on ASM (active shape model) [3] or AAM (active appearance model) [4]. Landmarks may be obtained therefrom which are useful for further processing. The landmarks may be calculated back by means of projection to obtain a normalized head and/or may be calculated back to the 3D on the basis of a normalized head. This may be effected, e.g., by means of POSIT (POS with ITerations) [5].

Following initialization, 2D reference points (feature points) may be searched and tracked (feature tracking) within the modeled face. Said reference points may but need not match the above-described landmarks. Alternatively or additionally, however, any deviating reference points will also be determined since the reference points frequently do not match the landmarks. On the basis of the initial position of the head as well as on the tracking of the reference points, position tracking of the head may be effected. To simplify matters, e.g. to reduce the computing expenditure, the surface of the head may be broken down to a 3D cylinder which is initialized with the previously determined initial position of the head. In other words, the 3D head modeler 110 may calculate an estimation of the three-dimensional position and orientation of the head, i.e. of a 3D head pose, on the basis of a cylinder.

For further tracking, 2D feature points are associated with corresponding 3D positions on the cylinder. This results in a spatial position and orientation in six degrees of freedom (6 DOF) of the scatter plot. The 2D feature points are tracked over time, i.e. from picture to picture, and their spatial positions are determined. If any feature points fall away, or if new feature points are added, for example due to a turning of the head, their spatial positions are determined on the basis of the 3D cylinder model. If too many 2D feature points are lost, the position of the 3D cylinder cannot be tracked any longer, and renewed initialization may be performed. The number of times a renewed initialization may be performed depends on the robustness of the 2D feature points and on the tracking of the feature points. The robustness, or susceptibility, of the feature points errors is dependent, e.g., on changes in the illumination, on perspective distortions or short-term instances of being obscured. In order to counteract temporal drifts (slow shifts), an adjustment with the landmarks and/or the 3D position obtained from the initialization may be performed now and then, i.e. one passes through initialization from time to time in parallel with the tracking. Moreover, the landmarks obtained from the initialization may be carried along in 3D in the tracking of the head's position, and their 2D determination may be found and used for said adjustment.

Further embodiments show the device 5 comprising an eye opening detector 130 which determines a determination and/or a time curve of the eye opening, expressed in normalized values, e.g. pixels normalized to the distance from the camera. In other words, the eye opening detector 130 constantly determines a current width of the eye opening. For analyzing the eyes, one or more landmarks within the ROI of the eye(s) are used. In case there is one single landmark, the position stemming from the initial eye recognition (see functional blocks of facial recognition 115 and/or eye recognition 120) may be used which was determined by using the cascade detector, for example. If only one single landmark is available, it will be advantageous for this landmark to be the eye center. Further important landmarks represent the corners of the eye as well as the upper and lower eyelids, for example. However, said landmarks are optional and may be used in support of an eye analysis being performed on the part of the eye opening detector. This has the advantage that the eye analysis may also be performed in case 3D head pose tracking fails and no landmarks can be provided and, thus, only an estimated eye position, e.g. the eye center, may be obtained from the 2D tracking of the functional blocks 115, 120 or 125. According to this, the eye opening detector 130 is configured to determine an eye opening on the basis of one or more landmarks stemming from the functional blocks 110 to 125. Determination of the eye opening is performed with the aid of a template. The eye opening detector 130 may model the eye opening by means of a template described across four points, or landmarks, and is adapted to the detected eye via the former. The edge region between the four landmarks is interpolated by means of mathematical curves. The eye opening detector thus is configured to determine the eye opening angle by means of a curve approximation which interpolates between at least four parameters describing the eye contour. Moreover, the template may be distorted in terms of perspective prior to being spanned, provided that the items of information about the 3D position and orientation of the head which stem from the functional block 110 are available.

In the following, two methods of determining the degree of eyelid closure, or the eye opening, will be described wherein the template is adjusted to the underlying picture content. Said adjustment, or fine adjustment, is effected via the gradient picture and other suitable features such as corner features or arch features, as was already described above.
1. Processing with the eye center as the only landmark: If the eye center is the sole landmark, an upper point and a lower point of the eye opening may be estimated, e.g., with the aid of a horizontal projection function (following [6], for example). The previously described template is spanned across the eye center and the upper and lower points of the eye opening, the width of the template depending on the width of the face.
2. Processing with several landmarks in the ROI of the eyes: The template is spanned, in accordance with the landmarks, within the area of the eyes, i.e., the ROI of the eyes. This is followed by orienting and adapting the template to the eye opening. The orientation and adaptation of the template may be effected, e.g., on the basis of the similarity to the gradient picture.

Moreover, the movement of the vehicle, e.g., in the field of automobiles, may be integrally measured via an acceleration sensor. During driving, accelerations occur due to accelerating, braking, different inclinations of the road and in curves. Vibration of the car during standstill and/or in a stationary state may be filtered out, for example by filtering a harmonic oscillation. In addition or as an alternative, a GPS (Global Positioning System) may be employed for detecting the movement of the vehicle. This is advantageous if the momentary-sleep warning is performed only if the vehicle is in movement (false alarms in a stationary vehicle are thus avoided). Moreover, the vehicle's speed may be used for finally adjusting the temporal adaptive threshold 85 for outputting a momentary-sleep warning.

The described device 5 relates to a camera-based and (with regard to the user) contactless system, the camera and/or video recording device 10 of which is directed at a person 15. FIG. 4 shows this scenario with a video recording device 10 directed at the person 15. The person may be the driver of a vehicle, for example. Moreover, the device 5 is suited to determine the individually adapted thresholds 80 and 85 as well as the individually determined reference values 70 and 75 for detecting that the person has momentarily fallen asleep in a camera live picture and to warn the person 15 in case momentary sleep has occurred. The term momentary-sleep warning device is to be understood as a term used in this invention rather than as an exact demarcation since it recognizes microsleep, momentary sleep as well as the occurrence of ongoing sleep.

The method and the algorithms used are characterized in that momentary sleep occurring in the person is recognized by means of direct video-based monitoring and of an evaluation which individually adapts to the person and the lighting conditions. As compared to the existing methods, which determine secondary data regarding the sleep state of the person, such as information present on the CAN bus within the vehicle, for example, via the steering, braking and accelerating behavior, primary data (eye opening) is evaluated. As a result, the system and/or the algorithms are able to recognize momentary sleep, and output a warning in case it occurs, in a clearly more accurate and reliable manner, as a result of which accidents may be avoided and people's lives may be saved in particular in safety-critical areas (e.g., driving of a vehicle, monitoring of aviation areas (e.g., on the part of air traffic controllers), operation and monitoring of power plants).

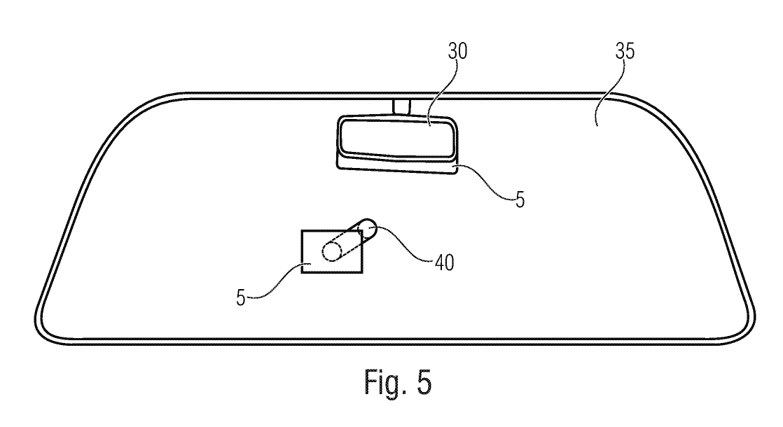

With regard to the automotive sector, the device 5 may be integrated into the rear-view mirror 30 within a car or may alternatively be mounted to a fresh-air grille or to the windshield, e.g., as a retrofit solution (such as a retrofit navigation device). FIG. 5 shows two of said arrangements, the device 5 being integrated into the rear-view mirror 30 and thus also being mounted to a windshield 35, just like the device 5, which is also shown, is mounted to the windshield 35 as a retrofitting solution by means of a fixture 40 (e.g., a suction cup fixture). Also, integration into the dashboard, the A-pillar or underneath the roof of the car is possible. As was already described, however, the device is not limited to the automotive sector but may also be employed in other areas. Moreover, a position from which the device is directed at the face from below at a slightly oblique angle is advantageous since, in this position, the device mostly has a clear view of the eyes, which are only rarely obscured by the eye sockets or eyebrows, for example.

The device 5 may be implemented as an autonomous device and may also be implemented, according to one embodiment, to be portable or to be part of a superordinated assistance or monitoring system. As a simple variant, the device may be used as a plug-and-play solution, i.e., installation may be performed by anyone. In this context, however, care is to be taken to ensure that the video recording device is directed at the driver. In addition to detecting the person 15 to be monitored, e.g., a car driver, a truck driver or an air traffic controller, by a camera, i.e., the video recording device 10, it is also possible to realize a system having several cameras. This may increase robustness in that the detection area (with one particular camera, one eye may be obscured which will then be captured by a second camera positioned in a different location) is expanded, or additional parameters (e.g., the line of vision of the person 15) are also taken into account in the evaluation.

In order to be independent of external illumination conditions, the illumination employed may be within the spectral range which is not visible to humans and therefore does not represent a disturbance, e.g., within the near-infrared range. Moreover, the state of the system may be indicated, for example, by means of differently colored LEDs, and the warning in the event of momentary sleep having occurred may be effected via an acoustic signal.

Moreover, the above-described device 5 comprises the following advantages:
eyelid closure is determined by means of direct, contactless, video-based measurement and derivation therefrom of the individually adapted thresholds 80 and 85 and of the individually determined reference values 70 and 75 for detecting momentary sleep therefrom
camera is directly aimed at the driver, as a result of which the driver's momentary sleep is recognized by direct video monitoring
remote system:
   contactless measurement without impairing the driver
   no attachment to the driver's head or any other part of the body is required
methodical advantages:
   evaluation of primary data which is derived from the driver's face on the basis of video recordings
   utilization of alternative algorithms which replace the described overall functionality (recognition of momentary sleep/microsleep) or individual partial functions (e.g., facial recognition, removal of reflections, etc.)

Fields of application are the automotive sector, for example, as well as any other transport sectors involving buses, trains, ships, submarines, trucks, etc., or any other safety-relevant sectors, e.g., in power plants, for air traffic controllers, for traffic monitoring, etc. The device may be integrated into existing driver assistance systems or monitoring systems, provided that a camera directed at the user is made available. Alternatively or additionally, a momentary-sleep warning device may be sold as an autonomous system (self-contained hardware). Said system may be mounted at the target location (normally during a one-off operation) such that the user is captured by the camera. Mounting may be performed by qualified personnel, in particular if a connection with other systems is to be established as well, e.g. with the CAN bus in passenger cars. Mere mounting and directing at the user may be performed by anyone, for example by means of a suction cup fixture on the inside of a passenger car's windshield.

Embodiments show that the duration of winking which is individually recognized in the person monitored may be utilized for not evaluating said short eylid closure times as momentary sleep. “Spontaneous blinking” and/or “spontaneous eyelid closure” in this document is understood to mean winking, for example.

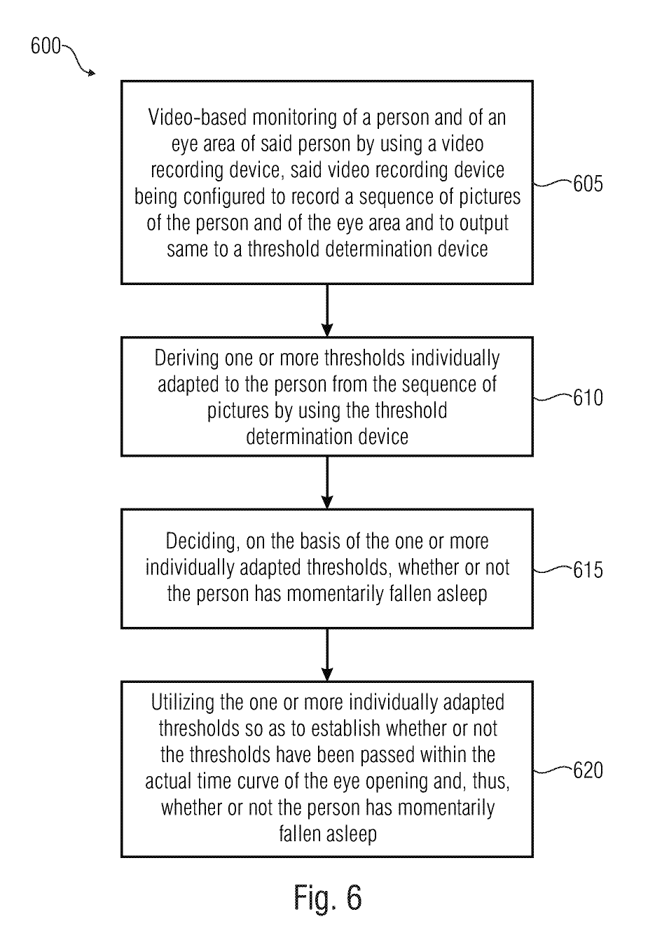

Further embodiments show a method 600 of detecting momentary sleep by means of steps of 605 “video-based monitoring of a person and of an eye area of said person by using a video recording device, said video recording device being configured to record a sequence of pictures of the person and of the eye area and to output same to a threshold determination device”, 610 “deriving one or more thresholds individually adapted to the person from the sequence of pictures by using the threshold determination device”, 615 “deciding, on the basis of the one or more individually adapted thresholds, whether or not the person has momentarily fallen asleep”, and 620 “utilizing the one or more individually adapted thresholds so as to establish whether or not the thresholds have been exceeded or fallen below within the actual time curve of the eye opening and, thus, whether or not the person has momentarily fallen asleep”. A schematic block diagram of the method is shown in FIG. 6.

Even though some aspects have been described within the context of a device, it is understood that said aspects also represent a description of the corresponding method, so that a block or a structural component of a device is also to be understood as a corresponding method step or as a feature of a method step. By analogy therewith, aspects that have been described within the context of or as a method step also represent a description of a corresponding block or detail or feature of a corresponding device. Some or all of the method steps may be performed by a hardware device (or while using a hardware device), such as a microprocessor, a programmable computer or an electronic circuit. In some embodiments, some or several of the most important method steps may be performed by such a device.

Depending on specific implementation requirements, embodiments of the invention may be implemented in hardware or in software. Implementation may be effected while using a digital storage medium, for example a floppy disc, a DVD, a Blu-ray disc, a CD, a ROM, a PROM, an EPROM, an EEPROM or a FLASH memory, a hard disc or any other magnetic or optical memory which has electronically readable control signals stored thereon which may cooperate, or actually do cooperate, with a programmable computer system such that the respective method is performed. This is why the digital storage medium may be computer-readable.

Some embodiments in accordance with the invention thus comprise a data carrier which comprises electronically readable control signals that are capable of cooperating with a programmable computer system such that any of the methods described herein is performed.

Generally, embodiments of the present invention may be implemented as a computer program product having a program code, the program code being effective to perform any of the methods when the computer program product runs on a computer.

The program code may also be stored on a machine-readable carrier, for example.

Other embodiments include the computer program for performing any of the methods described herein, said computer program being stored on a machine-readable carrier. In other words, an embodiment of the inventive method thus is a computer program which has a program code for performing any of the methods described herein, when the computer program runs on a computer.

A further embodiment of the inventive methods thus is a data carrier (or a digital storage medium or a computer-readable medium) on which the computer program for performing any of the methods described herein is recorded.

A further embodiment of the inventive method thus is a data stream or a sequence of signals representing the computer program for performing any of the methods described herein. The data stream or the sequence of signals may be configured, for example, to be transferred via a data communication link, for example via the internet.

A further embodiment includes a processing means, for example a computer or a programmable logic device, configured or adapted to perform any of the methods described herein.

A further embodiment includes a computer on which the computer program for performing any of the methods described herein is installed.

A further embodiment in accordance with the invention includes a device or a system configured to transmit a computer program for performing at least one of the methods described herein to a receiver. The transmission may be electronic or optical, for example. The receiver may be a computer, a mobile device, a memory device or a similar device, for example. The device or the system may include a file server for transmitting the computer program to the receiver, for example.

In some embodiments, a programmable logic device (for example a field-programmable gate array, an FPGA) may be used for performing some or all of the functionalities of the methods described herein. In some embodiments, a field-programmable gate array may cooperate with a microprocessor to perform any of the methods described herein. Generally, the methods are performed, in some embodiments, by any hardware device. Said hardware device may be any universally applicable hardware such as a computer processor (CPU), or may be a hardware specific to the method, such as an ASIC.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

### SOURCES

[1] Timm, Barth (2011): Accurate Eye Centre Localisation by means of Gradients
[2] Viola and Jones, “Rapid object detection using a boosted cascade of simple features”, Computer Vision and Pattern Recognition, 2001
[3] Tim F. Cootes, Chris J. Taylor: Active Shape Models—“Smart Snakes”. In: David Hogg u. a. (Hrsg.): BMVC92. Proceedings of the British Machine Vision Conference; 22.-24. September 1992, Leeds. Springer-Verlag, Berlin 1992, ISBN 3-540-19777-X, pp. 266-275.
[4] T. F. Cootes, G. J. Edwards, C. J. Taylor: “Active Appearance Models”, in: IEEE Transactions on Pattern Analysis and Machine Intelligence, Vol. 23, No. 6, June 2001, pp. 681-685
[5] DeMenthon, Daniel F., and Larry S. Davis. “Model-based object pose in 25 lines of code.” Computer Vision—ECCV'92. Springer Berlin Heidelberg, 1992.
[6] Zhi-Hua Zhou, Xin Geng: “Projection functions for eye detection.” Pattern Recognition, Volume 37, Issue 5, May 2004, pp. 1049-1056

1. A device for detecting momentary sleep, comprising:
a video recording device for video-based monitoring of a person and of an eye area of said person, the video recording device being configured to record a sequence of pictures of the person and of the eye area and to output same to a threshold determination device;
the threshold determination device configured to derive one or more thresholds individually adapted to the person from the sequence of pictures; and
a threshold evaluator configured to decide, on the basis of the one or more individually adapted thresholds, whether or not the person has momentarily fallen asleep;
the one or more individually adapted thresholds being utilized for establishing whether or not the thresholds have been passed within the actual time curve of the eye opening and, thus, whether or not the person has momentarily fallen asleep;
a first individually adapted threshold characterizing the transition from the opened to the closed eye, an eyelid closure being established to have occurred if the first individually adapted threshold is passed;
a second individually adapted threshold referring to a duration of the eyelid closure, an individual eyelid closure time being determined so as to evaluate the eyelid closure as being an indication of momentary sleep if the individual eyelid closure time is exceeded.

2. The device as claimed in claim 1, wherein the individually adapted thresholds are utilized in combination with a specific pupil center for detecting momentary sleep.

3. The device as claimed in claim 1, wherein a first individually adapted threshold is used for determining eyelid closure of an eye, said eyelid closure lying between a first individually determined reference value describing an opened eye and a second individually determined reference value describing a closed eye, and the threshold characterizing the transition from the opened to the closed eye;
a second individually determined threshold relating to a duration of the eyelid closure.

4. The device as claimed in claim 1, wherein the threshold determination device is configured to continually adapt the individually determined reference values and the one or more individually determined thresholds on the fly by evaluating the blinks that occur.

5. The device as claimed in claim 4, wherein the threshold determination device is configured to determine a blink by means of a pattern in the time curve of the eye opening, the pattern comprising, starting from the first individually determined reference value, falling below the first individually adaptive threshold as well as a return to the first reference value within a predefined time period.

6. The device as claimed in claim 1, wherein the threshold evaluator is configured to detect a first threshold having been passed and to determine a duration of said passage, the momentary sleep being established if the duration passes a second threshold.

7. The device as claimed in claim 1, wherein the threshold determination device is configured to adapt the one or more individually adapted thresholds to the anatomy and physiology of the person.

8. The device as claimed in claim 1, wherein the threshold determination device is configured to adapt the one or more individually adapted thresholds to the changed ambient conditions on the basis of a change in the ambient conditions.

9. The device as claimed in claim 1, wherein the first individually adapted threshold comprises a hysteresis configured to define eyelid closure at a relatively small eye opening angle during a transition from the opened to the closed eye and to define it at a relatively large eye opening angle during transition from the closed to the opened eye.

10. The device as claimed in claim 1, wherein the threshold determination device is configured to determine the one or more individually adapted thresholds separately for each eye.

11. The device as claimed in claim 1, wherein the threshold determination device comprises determining a head pose, the threshold determination device being configured to adapt the one or more individually adapted thresholds by means of the head pose determined.

12. The device as claimed in claim 11, wherein the threshold evaluator is configured to determine whether or not the person has momentarily fallen asleep by means of one eye or by means of both eyes of the person as a function of the head pose.

13. The device as claimed in claim 1, wherein a second individually adapted threshold indicates a duration longer than that of the winking.

14. The device as claimed in claim 1, wherein a second individually adapted threshold is adapted to the current speed at which the person is travelling.

15. The device as claimed in claim 1, the device comprising a GPS receiver and/or an acceleration sensor configured to capture a speed of travel of the device, conclusions being drawn from said speed of travel as to a speed of the person and/or of a vehicle.

16. The device as claimed in claim 1, the device comprising a source of illumination emitting radiation above a wavelength range visible to the person, the video recording device being configured to detect the radiation.

17. The device as claimed in claim 16, the device comprising a further source of illumination arranged at a distance from the source of illumination;
wherein the video recording device is configured to determine a combination of pictures taken in which the person is illuminated by subsequent illumination of the eye area by the source of illumination and the further source of illumination so as to avoid any reflections in the region of the eye area of the person.

18. The device as claimed in claim 1, the device being portable.

19. A method of detecting momentary sleep, comprising:
video-based monitoring of a person and of an eye area of said person by using a video recording device, said video recording device being configured to record a sequence of pictures of the person and of the eye area and to output same to a threshold determination device;
deriving one or more thresholds individually adapted to the person from the sequence of pictures by using the threshold determination device;
deciding, on the basis of the one or more individually adapted thresholds whether or not the person has momentarily fallen asleep; and
utilizing the one or more individually adapted thresholds so as to establish whether or not the thresholds have been passed within the actual time curve of the eye opening and, thus, whether or not the person has momentarily fallen asleep;
a first individually adapted threshold characterizing the transition from the opened to the closed eye, an eyelid closure being established to have occurred if the first individually adapted threshold is passed;
a second individually adapted threshold referring to a duration of the eyelid closure, an individual eyelid closure time being determined so as to evaluate the eyelid closure as being an indication of momentary sleep if the individual eyelid closure time is exceeded.

20. A non-transitory digital storage medium having a computer program stored thereon to perform the method of detecting momentary sleep, said method comprising:
video-based monitoring of a person and of an eye area of said person by using a video recording device, said video recording device being configured to record a sequence of pictures of the person and of the eye area and to output same to a threshold determination device;
deriving one or more thresholds individually adapted to the person from the sequence of pictures by using the threshold determination device;
deciding, on the basis of the one or more individually adapted thresholds whether or not the person has momentarily fallen asleep; and
utilizing the one or more individually adapted thresholds so as to establish whether or not the thresholds have been passed within the actual time curve of the eye opening and, thus, whether or not the person has momentarily fallen asleep;
a first individually adapted threshold characterizing the transition from the opened to the closed eye, an eyelid closure being established to have occurred if the first individually adapted threshold is fallen below;
a second individually adapted threshold referring to a duration of the eyelid closure, an individual eyelid closure time being determined so as to evaluate the eyelid closure as being an indication of momentary sleep if the individual eyelid closure time is exceeded,
when said computer program is run by a computer.
